(19) United States
(12) Patent Application Publication
(10) Pub. No.: US 2026/0217817 A1
(43) Pub. Date: Jul. 30, 2026

(54) METHODS FOR THE TREATMENT OF NONINFECTIOUS UVEITIS

(71) Applicant: Tourmaline Bio, Inc., New York, NY (US)

(72) Inventors: Sandeep KULKARNI, New York, NY (US); Yung CHYUNG, New York, NY (US); Susan Dana JONES, New York, NY (US); Ryan IARROBINO, New York, NY (US); W. Bradford MIDDLEKAUFF, New York, NY (US); Kristine ERICKSON, New York, NY (US)

(21) Appl. No.: 18/721,038

(22) PCT Filed: Dec. 6, 2023
(86) PCT No.: PCT/US2023/082706

§ 371 Date: Jun. 17, 2024

Related U.S. Application Data
(60) Provisional application No. 63/387,788, filed on Dec. 16, 2022.

(51) Int. Cl. C07K 16/24; A61K 31/56; A61K 39/395; A61P 27/02; C07K 16/28

(52) U.S. Cl. CPC C07K 16/248; A61K 31/56; A61K 39/3955; A61P 27/02; C07K 16/2866; C07K 2317/92

(57) ABSTRACT

The present disclosure provides methods of treating noninfectious uveitis comprising subcutaneously administering to a patient in need thereof a therapeutically effective dose of an anti-interleukin-6 (anti-IL-6) antibody or antibody fragment. Further provided herein are pharmacologically active agents, compositions, methods and/or dosing schedules for the treatment of noninfectious uveitis.

### CROSS-REFERENCE TO RELATED APPLICATIONS

This application claims priority to U.S. Provisional Patent Application No. 63/387,788, filed Dec. 16, 2022, which is incorporated by reference herein in its entirety for all purposes.

### DESCRIPTION OF THE TEXT FILE SUBMITTED ELECTRONICALLY

The contents of the electronic sequence listing (TOUR_001_01WO_SeqList_ST26.xml; Size: 14,172 bytes; and Date of Creation: Dec. 4, 2023) are herein incorporated by reference in their entirety.

### TECHNICAL FIELD

The disclosure relates to therapeutic antibody molecules and treatments for noninfectious uveitis (NIU).

### BACKGROUND

Uveitis is the inflammation of the uveal tract, the vascular layer between the sclera and the neuroretina, which can lead to significant visual impairment. Uveitis is the third leading cause of blindness in the United States and is often initially diagnosed in individuals of 20 to 50 years old. More than 160,000 people are diagnosed with uveitis in the United States each year.

Uveitis can be clinically divided into infectious uveitis and noninfectious uveitis. Infectious uveitis is caused by an immune response to fight an infection inside the eye. Noninfectious uveitis (NIU) can develop as an ocular symptom of a systemic disease. Most often, it is idiopathic. NIU accounts for approximately 80% of all uveitis cases. Macular edema (ME) is the most common sight-threatening complication in uveitis. NIU is complicated by macular edema (ME) in about 40% of cases.

Despite current treatments, uveitis remains a leading cause of blindness in the United States. Steroids and oral immunosuppressants continue to be a mainstay of therapy for treating patients with macular edema and inflammation associated with uveitis, but their long-term use is associated with harmful side effects. An estimated 20-30% of NIU patients are currently on biologics. However, approved biologics (TNF inhibitors) are still associated with high rates of treatment failure. Humira, a TNF blocking agent, is only modestly effective in treating NIU. Further, biologic disease-modifying antirheumatic drugs (DMARDs) are used off label in uveitis, which is disadvantageous in terms of insurance reimbursement.

There is a need in the art for an effective and well-tolerated treatment with fast onset of activity that is suitable for a longer-term, at-home dosing to reduce the potential for relapse.

### SUMMARY

Provided herein is a method of treating noninfectious uveitis (NIU) comprising administering to a patient in need thereof a therapeutically effective dose of an anti-interleukin-6 (anti-IL-6) antibody or antibody fragment having the variable heavy (VH) complementarity-determining regions (CDRs) as defined in SEQ ID NOs 2, 3 and 4, and the variable light (VL) CDRs as defined in SEQ ID NOs 8, 9 and 10.

In some embodiments, the anti-IL-6 antibody or antibody fragment comprises a heavy chain polypeptide comprising a polypeptide having at least about 98% identity to SEQ ID NO: 1 and a light chain polypeptide comprising a polypeptide having at least about 98% identity to SEQ ID NO: 7. In one aspect, the anti-IL-6 antibody or antibody fragment comprises a heavy chain polypeptide having the sequence of SEQ ID NO: 1 and a light chain polypeptide having the sequence of SEQ ID NO: 7.

In some embodiments, the anti-IL-6 antibody or antibody fragment containing said CDRs as described herein is contained in a pharmaceutical composition that comprises said anti-IL-6 antibody or antibody fragment and a pharmaceutically acceptable carrier.

In some embodiments, the therapeutically effective dose of the present disclosure is between 5 mg to 200 mg. In some embodiments, the therapeutically effective dose is about 5, about 7.5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 mg of the anti-IL-6 antibody or antibody fragment.

In some embodiments, therapeutically effective dose of the antibody, or an antigen binding fragment thereof may be administered by any suitable route including, but not limited to, oral, intravenous, intramuscular, intravitreal, suprachoroidal, subtenon, subconjunctival, intracameral, intra-arterial, intramedullary, intraperitoneal, intrathecal, intraventricular, transdermal, transcutaneous, topical, subcutaneous, intranasal, enteral, sublingual, hypospray, intravaginal or rectal routes. In one embodiment, the therapeutically effective dose is administered subcutaneously.

In some embodiments, the dosing schedules for the anti-IL-6 antibody or antibody fragment is every 1 week to every 24 weeks. In one embodiment, the therapeutically effective dose is administered every 4, 8, 12 or 24 weeks.

In one embodiment, the therapeutically effective dose of 100 mg is administered every 8 weeks. In another embodiment, the therapeutically effective dose of 50 mg is administered every 4 weeks. Still in another embodiment, the therapeutically effective dose of 50 mg is administered every 8 weeks.

In some embodiments, the treatment may be provided over a total duration of about 1 month, about 2 months, about 3 months, about 4 months, about 5 months, about 6 months, about 7 months, about 8 months, about 9 months, about 10 months, about 11 months, about 12 months, about 14 months, about 16 months, about 18 months, about 20 months, about 22 months or about 24 months.

In some embodiments, the method of the present disclosure comprises: (a) administering a loading dose of the anti-IL-6 antibody or antibody fragment to the patient for at least the first does during a loading regimen; and (b) thereafter administering a maintenance dose of the anti-IL-6 antibody or antibody fragment subcutaneously to the patient during a maintenance regimen. In some embodiments, the loading regimen comprises administering the loading dose every 1 week, every 2 weeks, or every 4 weeks. In some embodiments, the maintenance regimen comprises administering the maintenance dose every 4 weeks, every 8 weeks, every 12 weeks, or every 24 weeks. In some embodiments, the loading dose is greater than or equal to the maintenance dose. In some embodiments, the loading dose is less than the maintenance dose. In some embodiments, the loading dose is between 5 mg to 200 mg. In some embodiments, the maintenance dose is between 5 mg to 200 mg.

In some embodiments, the method of the present disclosure comprises: (a) administering at least one induction dose of the anti-IL-6 antibody or antibody fragment subcutaneously to the patient with an active uveitis flare such that an induction of remission is achieved; and (b) thereafter administering at least one maintenance dose of the anti-IL-6 antibody or antibody fragment subcutaneously to the patient such that the recurrence of the uveitis flare is prevented during a maintenance regimen.

In one embodiment, the loading regimen comprises one loading dose of 100 mg; and the maintenance regimen comprises the maintenance dose of 50 mg every 4 weeks for a total of at least 16 weeks followed 4 weeks after the loading dose. In another embodiment, the loading regimen comprises one loading dose of 50 mg; and the maintenance regimen comprises the maintenance dose of 50 mg every 8 weeks for a total of at least 16 weeks followed 4 weeks after the loading dose. Still in another embodiment, the loading regimen comprises one loading dose of 50 mg; and the maintenance regimen comprises the maintenance dose of 20 mg every 4 weeks for a total of at least 16 weeks followed 4 weeks after the loading dose. Still in another embodiment, the loading regimen comprises one loading dose of 20 mg; and the maintenance regimen comprises the maintenance dose of 10 mg every 4 weeks for a total of at least 16 weeks followed 4 weeks after the loading dose. Still in another embodiment, the loading regimen comprises one loading dose of 100 mg; and the maintenance regimen comprises the maintenance dose of 20 mg every 4 weeks for a total of at least 16 weeks followed 4 weeks after the loading dose.

In some embodiments, the method of the present disclosure further comprises: administering a steroid to the patient. In some embodiments, the steroid comprises prednisone or equivalent. In some embodiments, a therapeutically effective dosage of the steroid ranges between 10 mg and 100 mg daily. In some embodiments, a therapeutically effective dosage of the steroid is administered orally, parenterally, topically to the eye, via intravitreal implants, via intravitreal injections, or via suprachoroidal injections. In one embodiment, prednisone in the form of methyl prednisone is administered intravenously. In some embodiments, the steroid administration starts at least 2 days before the first dose of the anti-IL-6 antibody or antibody fragment administration. In some embodiments, the steroid administration is tapering at week 4 of the treatment. In some embodiments, the steroid administration is tapered off by week 15 of the treatment.

In some embodiments, the patient is at least 18 years old.

In some embodiments, the patient being treated in accordance with methods of the disclosure has macular edema (ME) due to noninfectious uveitis. In some embodiments, the patient has noninfectious intermediate, posterior, or pan-uveitis in the treatment eye. In one embodiment, uveitis is active. In another embodiment, the patient has ME in anterior uveitis that is unresponsive to topical steroids.

In some embodiments, the patient has stable immunomodulatory therapy (IMT) with no increase for at least four weeks before the first dose. In some embodiments, the patient has discontinued IMT other than the steroid at least 48 hours before the first dose. In one embodiment, the patient receives co-administration of IMT and anti-IL-6 antibody as described herein.

In some embodiments, the patient has central retinal thickness (CRT) or central subfield thickness (CST) of more than 300 μM. In some embodiments, the patient has best corrected visual acuity (BCVA) from at least 23 to no more than 70 Early Treatment Diabetic Retinopathy Study (ETDRS) letters or the Snellen equivalent. In some embodiments, the patient has best-corrected visual acuity of 20/400 or better in the fellow eye. In some embodiments, the patient has no evidence of malignancy, infection, or fibrosis by chest radiograph within three months before the treatment. In some embodiments, a female patient has a negative serum pregnancy test during the treatment period. In some embodiments, a male patient agrees to use barrier contraception when engaging in sexual activity during the treatment period and for 28 days after the last dose administration.

In some embodiments, the patient dose not experience:
(a) confirmed or suspected uveitis of infectious etiology or uveitis of traumatic etiology;
(b) confirmed or suspected central nervous system or ocular lymphoma;
(c) uncontrolled glaucoma;
(d) a significant ocular disease;
(e) an ocular or periocular infection;
(f) a history of herpetic infection;
(g) toxoplasmosis or toxoplasmosis scar;
(h) ocular malignancy;
(i) a systemic infection;
(j) immunocompromised;
(k) a history of gastrointestinal perforation or bleed; or
(l) allergy or hypersensitivity to the anti-IL-6 antibody or antibody fragment.

In some embodiments, the patient dose not receive:
(a) prior treatment with an anti-IL-6 or IL-6 receptor complex (IL-6R) antagonist therapy;
(b) a topical ocular medication other than lubricating eye drops;
(c) an implanted device;
(d) lens/media opacities or obscured ocular media;
(e) an intraocular surgery or treatment;
(f) capsulotomy within 6 months of the first dose.

In one embodiments, the anti-IL-6 or IL-6R antagonist therapy comprises tocilizumab and sarilumab.

In some embodiments, the patient is treatment-naïve or active despite corticosteroid or disease modifying anti-rheumatic drugs (DMARD) treatment.

In some embodiments, the method of treatment as described herein achieves one or more of the following results:
(a) a flare reduction;
(b) at least about 20% reduction in Central Retinal Thickness (CRT) or central subfield thickness (CST) in eyes with macular edema (CRT or CST >300 μm) when measured after the start of treatment;
(c) an increase in best corrected visual acuity (BCVA) of the patient by at least 10 letters when measured after the start of treatment; or
(d) quiescence with a total steroid dose of <10 mg/day.

In some embodiments, the flare reduction comprises (a) a 2-step reduction in vitreous haze in eyes with >1+ vitreous haze score (SUN scale) or a vitreous haze score of 0 in eyes with vitreous haze score of ≤1+ when measured after the start of treatment; (b) a reduction in retinal lesions when measured after the start of treatment; (c) a reduction in retinal vessel leakage; and (d) a reduction in anterior chamber cells.

In some embodiments, the probability of flare reduction from baseline is at least about 50%, about 60%, about 70%, about 80%, or about 90%. In some embodiments, wherein the probability of CRT or CST reduction from baseline is at least about 50%, about 60%, about 70%, about 80%, or about 90%. In some embodiments, the probability of achieving quiescence with a total steroid dose of <10 mg/day after the treatment is at least about 50%, about 60%, about 70%, about 80%, or about 90%. In some embodiments, the probability of receiving retreatment with the anti-IL-6 antibody or antibody fragment is no more than about 50%, about 40%, about 30%, about 20%, or about 10%. In another embodiment, CRT or CST is reduced to <300 μM.

In some embodiments, the treatment result is achieved within 16 weeks, 12 weeks, 8 weeks, or 4 weeks. In some embodiments, the treatment result is achieved during a long-term treatment, wherein the long-term is more than 24 weeks.

In some embodiments, the methods described herein further comprise a step of treating a subject with an additional form of therapy. In some embodiments, the additional form of therapy comprises administering one or more therapeutic agent in addition to the anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, anti-IL17a antibody, and/or anti-TNF alpha antibody), a soluble receptor (e.g., soluble IL-1 receptor, soluble TNF-alpha receptor), and an anti-inflammatory agent (e.g., antimetabolites (e.g., azathioprine, methotrexate, and mycophenolate), calcineurin inhibitors (e.g., cyclosporine and tacrolimus), possibly alkylating agents (e.g., cyclophosphamide and chlorambucil), and mofetil).

In some embodiments, provided are pharmacologically active agents, compositions, methods and/or dosing schedules that have certain advantages compared to the agents, compositions, methods and/or dosing schedules that are currently used and/or known in the art, including the ability to dose less frequently or to administer lower doses to obtain equivalent effects in inhibiting IL-6 mediated signaling.

### BRIEF DESCRIPTION OF THE DRAWINGS

FIG. 1 presents the schematic for a Phase 2 randomized, dose-ranging study of TOUR006 with treatment extension in patients with noninfectious uveitis.

FIG. 2A and FIG. 2B illustrate the predicted percentage changes of CRP over the treatment period for population A (FIG. 2A) and population B (FIG. 2B), respectively, under the dose regimen of 50 mg LD, followed by 20 mg Q4W starting at 4 weeks.

FIG. 3A and FIG. 3B illustrate the predicted percentage changes of CRP over the treatment period for population A (FIG. 3A) and population B (FIG. 3B), respectively, under the dose regimen of 20 mg LD, followed by 10 mg Q4W starting at 4 weeks.

### DETAILED DESCRIPTION

Provided herein are methods of treating noninfectious uveitis (NIU) comprising subcutaneously administering to a patient in need thereof a therapeutically effective dose of an anti-interleukin-6 (anti-IL-6) antibody or antibody fragment.

Further provided herein are pharmacologically active agents, compositions, methods and/or dosing schedules for the treatment of NIU.

### Antibodies

Provided herein are antibodies and antigen-binding fragments thereof that specifically bind IL-6. Antibodies and antigen-binding fragments disclosed herein specifically bind human IL-6. In some embodiments, an antibody may be specific for only human IL-6 and may exhibit no non-human cross-reactivity.

As used herein, the term “antibody” refers to immunoglobulin (Ig) molecules and immunologically active portions or fragments of immunoglobulin molecules, i.e., molecules that contain an antigen-binding site that specifically binds (immunoreacts with) an antigen (e.g., IL-6). By “specifically binds” or “immunoreacts with” is meant that the antibody reacts with one or more antigenic determinants of the desired antigen and does not react with other polypeptides. In some embodiments, an antibody is said to specifically bind an antigen when it preferentially recognizes its target antigen in a complex mixture of proteins and/or macromolecules. In some embodiments, an antibody “specifically binds” IL-6 if the antibody binds IL-6 with greater affinity, greater avidity, more readily and/or for greater duration than it binds other polypeptides.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU numbering system. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgAQ1 or IgA2) or subclass. IgG, IgD, and IgE antibodies generally contain two identical heavy chains and two identical light chains and two antigen combining domains, each composed of a VH and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., IL-6). It has been shown that the antigen-binding function of an antibody can be performed by portions or fragments of a full-length antibody. Examples of binding fragments encompassed within the term “antigen binding portion” of an antibody include (i) a Fab fragment, a monovalent fragment consisting of the VL, VH, CL and CHI domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3 (1): 1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody fragment”. An antibody fragment generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is IL-6 and the second antigen is not IL-6. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-IL-6 antigen-binding portion disclosed herein is a Fab, a F(ab′)₂, a Fab′, a Fv, a scFv, a Fd, a single domain antibody, a single chain camelid antibody, a diabody, a triabody, a tetrabody or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity, and is equal to the dissociation constant K_{d}. (See, Davies et al. (1990) Annual Rev Biochem 59:439-473). An antibody or antigen-binding portion provided herein is said to specifically bind IL-6 when the equilibrium binding constant (K_{d}) is ≤10 μM, preferably ≤10 nM, more preferably ≤10 nM, and most preferably ≤100 μM to about 1 μM, as measured by assays such as radioligand binding assays or similar assays known to those skilled in the art.

In some embodiments, an anti-IL-6 antibody or antigen-binding portion provided herein is monovalent or bivalent and comprises a single or double chain. Functionally, the binding affinity of an antibody or antigen-binding portion may be within the range of about 10⁻⁵M to 10⁻¹² M. For example, the binding affinity of an antibody or antigen-binding portion is from about 10⁻⁶ M to 10-12 M, from about 10⁻⁷ M to 10⁻¹² M, from about 10⁻⁸ M to 10-12 M, from about 10⁻⁹ M to 10⁻¹² M, from about 10⁻⁵ M to 10-11 M, from about 10⁻⁶ M to 10-11 M, from about 10⁻⁷ M to 10⁻¹¹ M, from about 10⁻⁸ M to 10⁻¹¹ M, from about 10⁻⁹ M to 10⁻¹¹ M, from about 10⁻¹⁰ M to 10⁻¹¹ M, from about 10⁻⁵ M to 10⁻¹⁰ M, from about 10⁻⁶ M to 10⁻¹⁰ M, from about 10⁻⁷ M to 10⁻¹⁰ M, from about 10⁻⁸ M to 10⁻¹⁰ M, from about 10⁻⁹M to 10⁻¹⁰ M, from about 10⁻⁵ M to 10⁻⁹ M, from about 10⁻⁶ M to 10⁻⁹M, from about 10⁻⁷ M to 10⁻⁹ M, from about 10⁻⁸ M to 10⁻⁹ M, from about 10⁻⁵ M to 10⁻⁸ M, from about 10⁻⁶ M to 10⁻⁸ M, from about 10⁻⁷ M to 10⁻⁸ M, from about 10⁻⁵ M to 10⁻⁷ M, from about 10⁻⁶ M to 10⁻⁷M or from about 10⁻⁵ M to 10⁻⁶ M.

A human anti-IL-6 monoclonal antibody (PF-04236921) was described in U.S. Pat. No. 8,188,235, the content of which is incorporated herein by reference in its entirety. The human anti-IL-6 monoclonal antibody is a fully human immunoglobulin G2 monoclonal antibody that binds to human IL-6 and has a half-life of 36-51 days. In phase I trials in healthy volunteers and patients with rheumatoid arthritis (protocol B0151001, NCT00838565 and NCT01166555), intravenous and subcutaneous (SC) administration of the human anti-IL-6 monoclonal antibody (PF-04236921) was well tolerated and caused sustained suppression of C-reactive protein (CRP), a marker for inflammation that is transcriptionally controlled by IL-6. PF-04236921 has also been investigated in a phase II trial in patients with systemic lupus erythematosus (SLE; NCT01405196). While the study did not meet the primary end point, improvement was noted in the primary as well as key secondary end points with 10 mg. In 448 subjects who have received treatment with the anti-IL-6 antibody, 2 subjects (0.45%) have tested positive for the presence of anti-drug antibodies. Overall, the human anti-IL-6 monoclonal antibody demonstrated desirable pharmacokinetic (PK) and pharmacodynamic (PD) properties supporting sustained target inhibition, and low incidence of immunogenicity upon single and multiple dose administration (Danese et al., Gut 2019; 68:40-48; Li et al., Br J Clin Pharmacol. 2018 September; 84 (9): 2059-2074.).

The amino acid and nucleic acid sequences of the human anti-IL-6 antibody (TOUR006) are provided in Table 1.

TABLE 1
Amino acid and nucleic acid sequences of human anti-IL-6 antibody

| | Anti-IL-6 antibody (TOUR006) | |
|---|---|---|
| Antibody name | | SEQ |
| Domain or Region | Sequence | ID NO |
| Heavy chain | QVQLQQWGAGLLKPSETLSLTCAIYGGSFREYYW | 1 |
| (amino acid | SWIRQPPGKGLEWIGEIFHSGSTNYNPSLKSRVTIS | |
| sequence) | VDTSKNQFSLKLSSVTAADTAVYYCAREELDDFD | |
| | IWGQGTMVTVSSASTKGPSVFPLAPCSRSTSESTA | |
| | ALGCLVKDYFPEPVTVSWNSGALTSGVHTFPAVL | |
| | QSSGLYSLSSVVTVPSSNFGTQTYTCNVDHKPSNT | |
| | KVDKTVERKCCVECPPCPAPPVAGPSVFLFPPKPK | |
| | DTLMISRTPEVTCVVVDVSHEDPEVQFNWYVDG | |
| | VEVHNAKTKPREEQFNSTFRVVSVLTVVHQDWL | |
| | NGKEYKCKVSNKGLPAPIEKTISKTKGQPREPQVY | |
| | TLPPSREEMTKNQVSLTCLVKGFYPSDIAVEWESN | |
| | GQPENNYKTTPPMLDSDGSFFLYSKLTVDKSRWQ | |
| | QGNVFSCSVMHEALHNHYTQKSLSLSPGK | |
| Heavy chain CDR1 | EYYWS | 2 |
| Heavy chain CDR2 | EIFHSGSTNYNPSLKS | 3 |
| Heavy chain CDR3 | EELDDFDI | 4 |
| Heavy chain | QVQLQQWGAGLLKPSETLSLTCAIYGGSFREYYW | 5 |
| variable region | SWIRQPPGKGLEWIGEIFHSGSTNYNPSLKSRVTIS | |
| (VH) (amino acid | VDTSKNQFSLKLSSVTAADTAVYYCAREELDDFD | |
| sequence) | IWGQGTMVTVSS | |
| Heavy chain | caggtgcagctacagcagtggggcgcaggactgttgaagccttcggagacc | 6 |
| (nucleic acid | ctgtccctcacctgcgctatctatggtgggtccttcagggagtactactggagct | |
| sequence) | ggatccgccagcccccagggaaggggctggagtggattggggaaatctttca | |
| | tagtggaagcaccaactacaacccgtccctcaagagtcgagtcaccatatcag | |
| | tagacacgtccaagaaccagttctccctgaagctgagctctgtgaccgccgcg | |
| | gacacggctgtgtattactgtgcgagagaggaattagatgattttgatatctggg | |
| | gccaagggacaatggtcaccgtctcctcagcctccaccaagggcccatcggt | |
| | cttccccctggcgccctgctccaggagcacctccgagagcacagcggccctg | |
| | ggctgcctggtcaaggactacttccccgaaccggtgacggtgtcgtggaactc | |
| | aggcgctctgaccagcggcgtgcacaccttcccggctgtcctacagtcctcag | |
| | gactctactccctcagcagcgtagtgaccgtgccctccagcaactteggcacc | |
| | cagacctacacctgcaacgtagatcacaagcccagcaacaccaaggtggac | |
| | aagacagttgagcgcaaatgttgtgtcgagtgcccaccgtgcccagcaccac | |
| | ctgtggcaggaccgtcagtcttcctcttccccccaaaacccaaggacaccctc | |
| | atgatctcccggacccctgaggtcacgtgcgtggtggtggacgtgagccacg | |
| | aagaccccgaggtccagttcaactggtacgtggacggcgtggaggtgcataa | |
| | tgccaagacaaagccacgggaggagcagttcaacagcacgttccgtgtggtc | |
| | agcgtcctcaccgtcgtgcaccaggactggctgaacggcaaggagtacaagt | |
| | gcaaggtctccaacaaaggcctcccagcccccatcgagaaaacc.atctcca | |
| | aaaccaaagggcagccccgagaaccacaggtgtacaccctgcccccatccc | |
| | gggaggagatgaccaagaaccaggtcagcctgacctgcctggtcaaaggctt | |
| | ctaccccagcgacatcgccgtggagtgggagagcaatgggcagccggaga | |
| | acaactacaagaccacacctcccatgctggactccgacggctccttcttcctct | |
| | acagcaagctcaccgtggacaagagcaggtggcagcaggggaacgtcttct | |
| | catgctccgtgatgcatgaggctctgcacaaccactacacgcagaagagcctc | |
| | tccctgtctccgggtaaa | |
| Light chain (amino | DIQMTQSPSSLSASVGDRVTITCRASQGISSWLAW | 7 |
| acid sequence) | YQQKPEKAPKSLIYAASSLQSGVPSRFSGSGSGTD | |
| | FTLTISSLQPEDFATYYCQQYKSYPRTFGQGTKVE | |
| | IKRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKS | |
| | FNRGEC | |
| Light chain CDR1 | RASQGISSWLA | 8 |
| Light chain CDR2 | AASSLQS | 9 |
| Light chain CDR3 | QQYKSYPRT | 10 |
| Light chain variable | DIQMTQSPSSLSASVGDRVTITCRASQGISSWLAW | 11 |
| region (VL) (amino | YQQKPEKAPKSLIYAASSLQSGVPSRFSGSGSGTD | |
| acid sequence) | FTLTISSLQPEDFATYYCQQYKSYPRTFGQGTKVE | |
| | IK | |
| Light chain (nucleic | gacatccagatgacccagtctccatcctcactgtctgcatctgtaggagacaga | 12 |
| acid sequence) | gtcaccatcacttgtcgggcgagtcagggtattagcagctggttagcctggtat | |
| | cagcagaaaccagagaaagcccctaagtccctgatctatgctgcatccagttt | |
| | gcaaagtggggtcccatcaaggttcagcggcagtggatctgggacagatttca | |
| | ctctcaccatcagtagcctgcagcctgaagattttgcaacttattactgccaa | |
| | cagtataaaagttaccctcggacgttcggccaagggaccaaggtggaaatcaa | |
| | acgaactgtggctgcaccatctgtcttcatcttcccgccatctgatgagcagt | |
| | tgaaatctggaactgcctctgttgtgtgcctgctgaataacttctatcccaga | |
| | gaggccaaagtacagtggaaggtggataacgccctccaatcgggtaactccca | |
| | ggagagtgtcacagagcaggacagcaaggacagcacctacagcctcagcagca | |
| | ccctgacgctgagcaaagcagactacgagaaacacaaagtctacgcctgcgaa | |
| | gtcacccatcagggcctgagctcgcccgtcacaaagagcttcaacagggga | |
| | gagtgt | |

CDR1, CDR2 and CDR3 (from left to right) sequences are underlined in the heavy chain and light chain, respectively.

Provided herein is a method of treating noninfectious uveitis comprising subcutaneously administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10. In some embodiments, said antibody or antibody fragment comprises a heavy chain polypeptide comprising a polypeptide having at least about 95%, about 96%, about 97%, about 98% or about 99% identity to SEQ ID NO: 1 and a light chain polypeptide comprising a polypeptide having at least about 95%, about 96%, about 97%, about 98% or about 99% identity to SEQ ID NO: 7. In some embodiments, said antibody or antibody fragment comprises a heavy chain polypeptide comprising a polypeptide having the sequence of SEQ ID NO: 1 and a light chain polypeptide comprising a polypeptide having the sequence of SEQ ID NO: 7. In some embodiments, the anti-IL-6 antibody or an antigen-binding portion comprises human IgG2 constant regions.

As used herein, the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 1 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4 |

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, about 75%, about 80%, about 82%, about 84%, about 85%, about 86%, about 87%, about 88%, about 89%, about 90%, about 91%, about 92%, about 93%, about 94%, about 95%, about 96%, about 97%, about 98%, about 99%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, considering the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman et al. ((1970) J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) is a BLOSUM 62 scoring matrix with a gap penalty of 12, a gap extend penalty of 4, and a frameshift gap penalty of 5.

In some embodiments, the percent identity between two amino acid or nucleotide sequences can be determined using the algorithm of Meyers et al. ((1989) CABIOS 4:11-17) which has been incorporated into the ALIGN program (version 2.0), using a PAM120 weight residue table, a gap length penalty of 12 and a gap penalty of 4.

In some embodiments, the anti-IL-6 antibody or antigen-binding portion provided herein is monoclonal.

In some embodiments, the anti-IL-6 antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1, IgG2, or IgG4 human constant region. Examples of chimeric antibodies and suitable techniques for their generation are provided in U.S. Pat. Nos. 4,816,567; 4,975,369; and 4,816,397, each of which is incorporated herein by reference in its entirety.

In some embodiments, the anti-IL-6 antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. In some embodiments, the VH domain, the VL domain, or both the VH domain and the VL domain of an anti-IL-6 antibody or antigen-binding portion provided herein comprise one or more human framework region amino acid sequences. In some embodiments, a humanized antibody comprises sequences that are entirely human except for the CDRs. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety.

In some embodiments, humanization comprises removal of post-translational modification (PTM) sites in the variable domain sequences (e.g., in the CDR or framework sequences) of a non-human antibody. For example, one or more PTM sites in CDR sequences may be removed by substituting certain amino acid residues. In some embodiments, humanization comprises CDR grafting and back mutation.

In some embodiments, the anti-IL-6 antibody or antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgAQ1 or IgA2. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU numbering system. In some embodiments, a position of an amino acid residue in a constant region of an immunoglobulin molecule is numbered according to EU nomenclature (Ward et al., 1995 Therap. Immunol. 2:77-94).

In some embodiments, the anti-IL-6 antibody or antigen-binding portion thereof may comprise an immunoglobulin light chain constant region that is a kappa light chain constant region or a lambda light chain constant region.

In some embodiments, the anti-IL-6 antibody or antigen-binding portion thereof may comprise a human IgG4 constant region comprising the amino acid substitution S228P and a kappa light chain constant region.

Further provided herein is an immunoconjugate comprising an anti-IL-6 antibody or an antigen-binding portion linked to a therapeutic agent. In some embodiments, the therapeutic agent is a small molecule drug.

### Pharmaceutical Compositions

The anti-IL-6 antibodies and antigen-binding portions described herein (also referred to herein as “active compounds”) can be incorporated into pharmaceutical compositions suitable for administration. TOUR006 may potentially be treated through home administration (either self-administered or by caregiver or by visiting healthcare professional). Such compositions typically comprise an anti-IL-6 antibody or antigen-binding portion (or an immunoconjugate comprising said antibody or portion), and a pharmaceutically acceptable carrier, diluent or excipient. As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Provided herein is a pharmaceutical composition comprising (i) an anti-IL-6 antibody or an antigen-binding portion thereof, wherein the antibody or antigen-binding portion comprises a VH domain and a VL domain, wherein: (a) the VH domain amino acid sequence comprises HCDR1 of SEQ ID NO: 2, HCDR2 of SEQ ID NO: 3 and HCDR3 of SEQ ID NO: 4; and the VL domain amino acid sequence comprises LCDR1 of SEQ ID NO: 8, LCDR2 of SEQ ID NO: 9 and LCDR3 of SEQ ID NO: 10; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, subconjunctival, subtenon, suprachoroidal, intracameral, intravitreal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the anti-IL-6 antibody of the present disclosure is formulated in a aqueous solution. In some embodiments, the aqueous solution comprises the anti-IL-6 antibody at a concentration ranging from about 50 mg/mL to about 150 mg/mL. In some embodiments, the aqueous solution comprises the anti-IL-6 antibody at a concentration of about 50 mg/mL, about 55 mg/mL, about 60 mg/mL, 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL about 120 mg/mL, about 125 mg/mL, about 130 mg/mL, about 135 mg/mL, about 140 mg/mL, about 145 mg/mL, or about 150 mg/mL.

In some embodiments, a buffer is selected from phosphate buffers, histidine, sodium citrate, HEPES, Tris, Bicine, glycine, N-glycylglycine, sodium acetate, sodium carbonate, glycylglycine, lysine, arginine, sodium phosphate, and any combination thereof. Exemplary concentrations of buffers for formulations of the present disclosure are from about 5 mM to about 100 mM, about 50 mM, about 10 mM to about 40 mM, or about 20 mM. In some embodiments, histidine is included at about 5 mM, about 10 mM, about 20 mM, about 30 mM, about 40 mM, or about 50 mM.

In some embodiments, a sweetening agent is selected from sucrose and saccharin. In some embodiments, sucrose is included at about 50 mg/mL, about 60 mg/mL, about 80 mg/mL, about 90 mg/mL, or about 100 mg/mL.

In some embodiments, a tonicity adjusting agent is selected from sodium chloride, potassium chloride, dextrose, mannitol, glycerin, sorbitol, and any combination thereof. In some embodiments, mannitol is included at about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL.

In some embodiments, a chelating agents is selected from ethylenediaminetetraacetic acid (EDTA), disodium edetate, calcium EDTA, and any combination thereof. In some embodiments, EDTA is included at about 0.01 mg/mL, about 0.02 mg/mL, 0.03 mg/mL, about 0.04 mg/mL, 0.05 mg/mL, about 0.06 mg/mL, 0.07 mg/mL, about 0.08 mg/mL, about 0.09 mg/mL, or 0.10 mg/mL.

In some embodiments, a surfactant is selected from polysorbate 80, sodium lauryl sulfate (SDS), Tween 80, and any combination thereof. In some embodiments, polysorbate 80 is included at about 0.1 mg/mL, about 0.2 mg/mL, 0.3 mg/mL, about 0.4 mg/mL, 0.5 mg/mL, about 0.6 mg/mL, 0.7 mg/mL, about 0.8 mg/mL, about 0.9 mg/mL, or about 1.0 mg/mL.

In some embodiments, the formulation has a pH value ranging from about 5.0 to about 8.0. In some embodiments, the formulation has a pH value of about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0.

In some embodiments, TOUR006 is formulated at a concentration of 85 mg/mL with 20 mM histidine, 63.2 mg/mL sucrose, 16.8 mg/mL mannitol, 0.05 mg/mL EDTA, and 0.2 mg/mL polysorbate 80, pH 5.8. After reconstitution with water for injection, each single use vial contains 106 mg of TOUR006 in 1.25 mL of aqueous solution.

The pharmaceutical compositions provided herein can be included in a container, pack, or dispenser together with instructions for administration.

### Uses of Antibodies

Provided herein are methods and uses of the anti-IL-6 antibodies, anti-IL-6 antigen-binding portions, immunoconjugates and pharmaceutical compositions described herein for providing a therapeutic benefit to a subject with a condition associated with IL-6 expression. In some embodiments, the condition is noninfectious uveitis.

In some embodiments, the methods described herein further comprise a step of treating a subject with an additional form of therapy. In some embodiments, the additional form of therapy comprises administering one or more therapeutic agent in addition to the said anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, anti-IL17a antibody, and/or anti-TNF alpha antibody), a soluble receptor (e.g., soluble IL-1 receptor, soluble TNF-alpha receptor), and an anti-inflammatory agent (e.g., antimetabolites (e.g., azathioprine, methotrexate, and mycophenolate), calcineurin inhibitors (e.g., cyclosporine and tacrolimus), possibly alkylating agents (e.g., cyclophosphamide and chlorambucil), and mofetil).

Provided herein is an anti-IL-6 antibody or an anti-IL-6 antigen-binding portion, an immunoconjugate or a pharmaceutical composition described herein, for use as a medicament.

As used herein, the term “effective amount” or “therapeutically effective amount” refers to the amount of a pharmaceutical agent, e.g., an anti-IL-6 antibody or an antigen-binding portion thereof, which is sufficient to reduce or ameliorate the severity and/or duration of a disorder, e.g., noninfectious uveitis, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). In some embodiments, the therapeutically effective dose of said anti-IL-6 antibody or antibody fragment is effective to change one or more biomarkers of IL-6 mediated signaling including, but not limited to, total sIL-6R, total IL-6, C-reactive protein (CRP), an/or autoantibodies, for unexpectedly prolonged periods of time.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of an anti-IL-6 antibody according to the methods described herein. Pre-treatment does not exclude, and often includes, the prior administration of treatments other than an anti-IL-6 antibody.

As used herein, “post-treatment” means after the administration of an anti-IL-6 antibody according to the methods described herein. Post-treatment includes after any administration of an anti-IL-6 antibody at any dosage described herein. Post-treatment also includes after the treatment phase of an anti-IL-6 antibody.

As used herein, the terms “about” and “approximately” generally mean plus or minus 10% of the value stated. For example, about 0.5 would include 0.45 and 0.55, about 10 would include 9 to 11, about 1000 would include 900 to 1100.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann et al., 1991, Int. J. Cancer 47:659-664; Bagshawe et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 μg to 1 g for systemic applications, and 1 μg to 1 mg for intradermal, intravitreal, subconjunctival, subtenon, suprachoroidal, intracameral injection. In one embodiment, an initial higher loading dose, followed by one or more lower doses, may be administered. In another embodiment, an initial lower loading dose, followed by one or more higher doses, may be administered. In some embodiments, the antibody is a whole antibody, e.g., the IgG1, IgG2 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated. In some embodiments, the dosing of the present disclosure comprises an amount of at least about 10 mg, or at least about 20 mg, or at least about 30 mg, or at least about 40 mg, or at least about 50 mg of the anti-IL-6 antibody or antibody fragment.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above. In some embodiments, the dosing schedules for the anti-IL-6 antibody or antibody fragment is once every 4 or 8 weeks up to about 52 total weeks.

In some embodiments, a subject is a human, a non-human primate, a pig, a horse, a cow, a dog, a cat, a guinea pig, a rabbit, a mouse or a rat. In some embodiments, a subject is an adult human. In some embodiments, a subject is a pediatric human.

### Noninfectious Uveitis

As used herein, “uveitis” refers to inflammation of the uveal tissues of the eye, including the iris, ciliary body, and choroid. Other intraocular structures can also be involved in uveitis, including the sclera (termed scleritis), retina, retinal blood vessels, and the optic nerve. Uveitis is categorized as infectious or noninfectious. Noninfectious uveitis (NIU) can occur with systemic autoimmune disease and autoimmune diseases localized to the eye as well as trauma.

The main signs of uveitis may include conjunctival redness, abnormal eye pressure, anterior chamber cells and flare, iris synechiae, vitreous cells and haze, macular edema, retinal lesions, retinal vasculitis, and retinal and choroidal blood vessel leakage. Symptoms include light sensitivity, eye pain, blurred vision, and floaters

As used herein, “cell”, “flare”, and “haze” are standard descriptive terms. “Cell” refers to the number of inflammatory cells seen in a specific eye chamber. “Flare” is a term distinct to the anterior chamber that refers to the fog-like effect caused by protein exudation from vascular leakage in inflammation. “Haze” is a term specific to the vitreous cavity that refers to the haze caused by protein exudation and inflammatory cells in the vitreous humor.

There are several classifications of uveitis. According to the SUN working group definition, uveitis is classified by the anatomic location. A further classification based on etiology is noninfectious and infectious.

Anterior uveitis involves inflammation in the eye's anterior segment. It presents as an iritis or iridocyclitis. The anterior ocular segment consists of the anterior chamber, and the posterior chamber. The anterior chamber is bounded by the corneal endothelium and the anterior surface of the iris. The posterior chamber is bounded by the posterior surface of the iris and the anterior hyaloid membrane of the vitreous and includes the lens and the ciliary body. Clinical signs and symptoms can include anterior chamber cells and flare, keratic precipitates, pupillary changes, anterior and posterior synechiae, redness, pain, blurred vision, light sensitivity, and floaters. Noninfectious anterior uveitis can result from trauma or be associated with systemic conditions including HLA-B27 related disorders, sarcoidosis, juvenile ideopathic arthritis, and inflammatory bowel disease. Many cases are idiopathic with no known cause.

In noninfectious intermediate uveitis, the vitreous humor is the primary site of inflammation. Although over 70% of intermediate uveitis is idiopathic (pars planitis), identifiable causes include lymphoma (masquerade syndrome), multiple sclerosis and sarcoidosis. Pars planitis presents with “snowbanks” (white exudates over the pars plana and ora serrata) and “snowballs” (aggregates of inflammatory cells in the vitreous). A hallmark presentation of all intermediate uveitis is the presence of cells and haze in the vitreous humor. Clinical symptoms include blurry vision, floaters, and light sensitivity.

Noninfectious posterior uveitis is characterized by inflammation of the retina, retinal vessels, optic nerve head, choroid or choroidal vessels. This term encompasses both choroiditis, inflammation of deeper blood vessels, and retinitis, inflammation of the retina. Posterior uveitis may include macular edema, lesions of the retina, focal chorioretinal lesions, retinal ischemia, retinal detachments, retinal vasculitis, and optic nerve edema. While the majority of noninfectious posterior uveitis is idiopathic, known causes include punctate inner choroidopathy, Behcet's disease, sarcoidosis, and birdshot chorioretinopathy. Clinical symptoms include floaters and decreased vision. There is usually no pain or redness.

Noninfectious pan uveitis is defined as inflammation found within multiple ocular segments. Pan uveitis can be idiopathic or associated with systemic diseases such as sarcoidosis, Bechet's disease, or Vogt-Koyanagi-Harada disease. Clinical symptoms include reduced vision, floaters, pain, redness, and light sensitivity.

In addition to the anatomical descriptors defined above, the 2005 Standardization of Uveitis Nomenclature (SUN) Working Group also defined descriptors for onset, duration, and course. Onset can either be described as insidious or sudden. Duration is limited if the uveitis episode lasts less than 3 months and persistent if the episode lasts more than 3 months.

The combination of onset, duration, and recurrence defines the disease course. Acute course is one with sudden onset and limited duration. Chronic course is a persistent duration with recurrence within 3 months of treatment discontinuation. Recurrent course is characterized by repeated uveitis episodes separated by periods of inactivity greater than three months since treatment discontinuation.

The common findings of anterior chamber (AC) inflammation are cell and flare. The SUN Working Group defined their two AC grading schemas around these two findings. In general, an improvement in inflammation is a two-step decrease in grade. A worsening of inflammation occurs with a two-step increase in grade. Exceptions include an improvement in 0.5+ grading and a worsening of 3+ grading. For both, a one-step change would suffice.

The AC cell grading schema is an ordinal scale of 0 to 4+ increasing with the number of cells. The findings are standardized under a slit lamp beam of 1 mm×1 mm at the highest illumination. AC cell grading schema is provided in Table 2.

TABLE 2
Anterior chamber cell grading schema

0 cells in the field are graded as 0.
1 to 5 cells are graded as 0.5+, often called trace inflammation.
6 to 15 cells in the field are graded as 1+.
16 to 25 cells on the field are graded as 2+.
26 to 50 cells on the field are graded as 3+
Greater than 50 cells on the field are 4+

Grading anterior chamber flare follows a similar ordinal scale of 0 to 4+ with increasing flare. Whereas individual cells are counted for AC cell grading, a qualitative description of clarity of the iris and lens is used as the main metric. Anterior chamber flare grading schema is provided in Table 3.

TABLE 3
Anterior chamber flare grading schema

Grade of 0 is defined as no haze seen.
1+ grade presents with faint haze, and 2+ presents with moderate haze.
Grade of 3+ is defined as marked inflammation with hazy visualization of
the iris and lens.
Grade of 4+ has intense haze with fibrin or plastic aqueous seen at the slit
lamp.

Similar to AC grading, vitreous inflammation is graded on cells and haze in the vitreous humor. Uniquely, vitreous haze can present as a sign of intermediate uveitis and posterior or panuveitis. It often deteriorates vision far more significantly than anterior chamber inflammation. (Davis et al., Am J Ophthalmol 2010; 150:637).

The Multicenter Uveitis Steroid Treatment (MUST) Trial provided a vitreous cell grading scale. (Group TMUSTTR, Am J Ophthalmol 2010; 149:550). In a slit lamp field of 1 mm×0.5 mm, the cells were graded on an ordinal scale of 0 to 4+. Vitreous cell grading scale is provided in Table 4.

TABLE 4
Vitreous cell grading scale

A grade of 0 presents with no cells.
1 to 5 cells were seen in the field are graded as 0.5+.
6 to 10 cells are graded as 1+
11 through 20 cells are graded as 2+.
21 through 50 cells are given a grade of 3+
4+ is graded as fifty-one or more cells

Vitreous haze is analogous to the discussion of AC flare above. Similar to the SUN AC flare grading schema, the National Institute of Health (NIH) Scale grades vitreous haze based on visualization of anatomic landmarks compared to a set of standardized fundus photos. (Nussenblatt et al., Ophthalmology. 1985; 92 (4): 467-471). Like the other grading schemas discussed, it is an ordinal scale with grading from 0 to 4+. Vitreous haze grading scale is provided in Table 5.

TABLE 5
Vitreous haze grading scale

Normal findings of no haze are graded as 0.
A grade of 0.5+ or trace haze is defined as slight blurring of the optic disc
margin.
Obscured view with definition to the optic nerve head and retinal vessels is
given a grade of 1+. Obscured view with definition to the retinal vessels is
grade 2+ vitreous haze.
A grade of 3+ is given for the case where the optic nerve head is
visualized, but with blurry borders.
An obscured fundal view is defined as a grade 4+.

It is generally accepted that at least a two-step decrease in the level of inflammation for improvement and at least a two-step increase in the level of inflammation for worsening were better criteria than one-step changes. However, because of floor and ceiling effects (that is, 3+ only can increase by one step and 0.5+ only can decrease by one step), the definition of improvement should include a decrease in inflammation from 0.5+ to inactive, and the definition of worsening should include an increase from 3+ to the maximum grade. Hence, improvement in the inflammation will be defined as either a two-step decrease in the level of inflammation or a decrease to “inactive,” and worsening of the inflammation will be defined as either a two-step increase in the level of inflammation or an increase to the maximum grade. (The SUN working group, Am J Ophthalmol. 2005 September; 140 (3): 509-516.). The SUN Working Group activity of uveitis terminology is provided in Table 6.

TABLE 6
The SUN working group activity of uveitis terminology

| Term | Definition |
|---|---|
| Inactive | Grade 0 |
| Worsening | Two step increase in level of inflammation (e.g. anterior |
| activity | chamber cells, vitreous haze) or increase from grade 3+ to 4+ |
| Improved | Two step decrease in level of inflammation (e.g. anterior |
| activity | chamber cells, vitreous haze) or decrease to grade 0 |
| Remission | Inactive disease for ≥3 months after discontinuing all |
| | treatments for eye disease |

Uveitis activity may also be determined via reduction of the Best Corrected Visual Acuity (BCVA)/Early Treatment Diabetic Retinopathy Study (ETDRS) letter score or Snellen equivalent. For ETDRS, BCVA is determined using methodology adapted from the 4-meter Early Treatment Diabetic Retinopathy Study (ETDRS) protocol (using Early Treatment Diabetic Retinopathy Study (ETDRS) like charts) and resulting in the respective letter score. ETDRS charts present a series of five letters of equal difficulty on each row, with standardized spacing between letters and rows, for a total of 14 lines (70 letters). (Kniestedt C S R, Ophthalmol Clin North Am. 2003; 16 (2): 155-170). ETDRS letter score can be calculated when 20 or more letters are read correctly at 4.0 metres; the visual acuity letter score is equal to the total number of letters read correctly at 4.0 metres plus 30. If fewer than 20 letters are read correctly at 4.0 metres, the visual acuity letter score is equal to the total number of letters read correctly at 4.0 metres (number of letters recorded on line 1.0), plus the total number of letters in the first six lines read correctly at 1.0 metre. The most common method of measuring BCVA in clinical practice is the Snellen chart. The Snellen chart uses a geometric scale to measure visual acuity, with normal vision at a distance being set at 20/20. The numerator represents the distance that the patient is standing from the chart (in feet), while the denominator represents the distance from which a person with perfect eyesight is still able to read the smallest line that the patient can clearly visualize.

Macular edema occurs when fluid and protein deposits collect on or under the macula of the eye, the central area of the retina where the foveal centralis is located. The foveal centralis is responsible for sharp central vision (also called foveal vision). The thickening and swelling in macular edema may distort a person's central vision. This area holds tightly packed cones that provide sharp, clear central vision to enable a person to see form, color, and detail that is directly in the line of sight. The degree of macular thickening is significantly correlated with foveal visual vision acuity which is necessary for activities for which visual detail is of primary importance, such as reading and driving.

The retinal disease evaluation system of another aspect preferably includes evaluation of vision-related quality of life (QOL). The National Eye Institute Visual Function Questionnaire-25 (NEI VFQ-25) remains one of the most commonly used patient-reported outcome (PRO) measures in ophthalmology studies. The NEI VFQ-25 is constituted from visual functions in various living scenes, and 12 subscales (questions of 25 items) for measurement of the degree of restrictions of vision-related physical, mental and social living scenes (Mangione et al, Arch Ophthalmol. 2001). A blank questionnaire (National Eye Institute Visual Functioning Questionnaire-25 (VFQ-25) Version 2000-Interviewer Administered Format) with all questions is hereby incorporated by reference.

As used herein, the term “C-reactive protein (CRP)” refers to a marker of inflammation. CRP levels increase in response to inflammation, and can be measured with a hsCRP (high-sensitivity C-reactive protein) test. The pre-treatment hsCRP of the patients is typically greater than 2 mg/L. Under certain circumstances, the pre-treatment hsCRP level of the patient is 1 mg/L or less.

The treatment methods provided herein may achieve the following therapeutic responses.

### Cell and Flare Reduction

In one embodiment, the therapeutic response is a decrease in cell and flare, as measured by methods known to those of skill in the art. For examples, cell and flare reduction may be measured by 2-step reduction in anterior chamber cells and flare measured according to the SUN classification (see Table 2 and Table 3).

An increase or decrease in the number of anterior chamber cells can be indicative of improving or worsening disease and are critical in identifying active inflammation and rationalizing treatment decisions. The current standard measurement of the number of anterior chamber cells as defined by the SUN grading system is clinical examination by slit-lamp biomicroscopy, whereby a clinician aims a 1× 1-mm slit beam through the anterior chamber and counts the number of cells visible in the lit area (Jabs D A, Am J Ophthalmol. 2005; 140:509-16). The cell count is placed into one of six grades in the SUN grading system as described in Table 2.

### Vitreous Haze Reduction

Decrease in vitreous haze can be used as a measure of the method's efficacy. Decreases in vitreous haze can be qualitatively and/or quantitatively determined by validated clinical scales such as the SUN Scale or the Miami Scale utilizing techniques (Davis et al, Am J Ophthalmol. 2010; 150:637) such as, but not limited to, slit lamp evaluation, photographic grading by a reading center, a scoring system, a multi-point scale, a multi-step scale (e.g. a multi-step logarithmic scale, manual screening by one or more examiners, and/or the like).

In one embodiment, the decrease in vitreous haze is present about 2 weeks, about 1 month, about 2 months, about 3 months, about 4 months, about 5 months, or about 6 months after the at least one dosing session. In another embodiment, where multiple dosing sessions are employed, a decrease in vitreous haze is experienced by the patient and is present at least about 2 weeks, at least about 1 month, at least about 2 months, at least about 3 months, about 4 months, about 5 months, or at least about 6 months after each dosing session.

### Reduction in Central Retinal Thickness (CRT) or Central Retinal Subfield Thickness (CST) Reduction

Macular edema can be assessed by measurement of the central retina (central retinal thickness; CRT) or central retinal subfield thickness (CST) measured by optical coherence tomography (OCT) (Chan et al., Arch Ophthalmol. 2006 February; 124 (2): 193-198.). OCT has enabled clinicians to reliably detect and measure small changes in macular thickness and to quantitatively evaluate the efficacy of different therapeutic modalities. In one embodiment CRT or CST is measured by spectral domain OCT including Stratus OCT (Carl Zeiss Meditec).

In one embodiment, the therapeutic response is a change from baseline in CRT or CST at one or more time points after the patient is treated. For example, at one week, two weeks, three weeks, one month, two months, three months, four months or more, including all durations in between, after a dosing session. A decrease in CRT or CST (as compared to prior to treatment) is one measurement of therapeutic response (e.g., by about 10%, or about 20%, or about 30%, or about 40%, or about 50%, or about 60% and more, including all values in between, from baseline).

In one embodiment, the decrease in retinal thickness as measured by CRT or CST is present at least about 2 weeks, at least about 1 month, at least about 2 months, at least about 3 months or at least about 6 months after at least one dosing session.

In another embodiment, where multiple dosing sessions are employed, the decrease in retinal thickness as measured by CRT or CST is present at least about 2 weeks, at least about 1 month, at least about 2 months, at least about 3 months or at least about 6 months after at least one dosing session.

Reduction in Choroiditis and/or Retinitis as Measured by a Reduction in Choroidal, Retinal or Chorioretinal Lesions

“Retinal lesions”, “choroidal lesions” or “chorioretinal lesions” referred to herein, can be characterized by pale lesions with reasonably well demarcated borders, depigmented and hyperpigmented lesions surrounding the optic disc (“bullet-hole” or “butterfly” lesions), retinal detachment, puckering, fibrosis, lamellar splitting, and/or dragging, swelling, bulging of retinal tissues and retinal holes. Retinal lesions may be measured by methods known to those of skill in the art, including fundus photography, optical coherence tomography (OCT), visual acuity, or fundus examination. either locally or via a central reading center.

In one embodiment, the decrease in choroiditis and/or retinitis is present at least about 2 weeks, at least about 1 month, at least about 2 months, at least about 3 months or at least about 6 months after at least one dosing session.

In another embodiment, where multiple dosing sessions are employed, the decrease in choroiditis and/or retinitis is present at least about 2 weeks, at least about 1 month, at least about 2 months, at least about 3 months or at least about 6 months after at least one dosing session.

### Reduction in Retinal Vascular Leakage

In one embodiment, reduction or suppression in retinal vessel leakage is used as a measure of the method's efficacy. Retinal vessel leak is measured through fluorescein angiography (FA). In some embodiments, vascular leakage is suppressed over 2 weeks, more than 4 weeks, more than 8 weeks or more than 10 weeks since the starting of the treatment.

### BCVA Improvement

In some embodiments, efficacy is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after at least one dosing session, 3 months after administration), of at least 2 letters, at least 3 letters, at least 5 letters, at least 10 letters, at least 12 letters, at least 13 letters, at least 15 letters, at least 20 letters, and all values in between, as compared to the patient's BVCA prior to the at least one dosing session.

In one embodiment, the patient, for example with macular edema associated with uveitis gains about 5 letters or more, about 10 letters or more, 15 letters or more, about 20 letters or more, about 25 letters or more in a BCVA measurement after a dosing regimen is complete, for example a monthly dosing regimen, compared to the patient's BCVA measurement prior to undergoing treatment. In even a further embodiment, the patient gains from about 5 to about 30 letters, from about 10 to about 30 letters, from about 15 letters to about 25 letters or from about 15 letters to about 20 letters in a BCVA measurement upon completion of at least one dosing session, compared to the patient's BCVA measurement prior to the at least one dosing session. In one embodiment, the BCVA gain is about 2 weeks, about 1 month, about 2 months, about 3 months or about 6 months after the at least one dosing session. In another embodiment, the BCVA is measured at least about 2 weeks, at least about 1 month, at least about 2 months, at least about 3 months or at least about 6 months after the at least one dosing session.

In one embodiment, the BCVA is based on the Early Treatment of Diabetic Retinopathy Study (ETDRS) visual acuity charts and is assessed at a starting distance of 4 meters.

In another embodiment the BCVA is based on the Snellen chart or equivalent and is assessed at 20 feet.

In another embodiment, the patient subjected to a treatment method, e.g., with one of the devices provided herein substantially maintains his or her vision subsequent to the treatment (e.g., a single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

### Safety and Tolerability of a Therapy for NIU

Safety and tolerability of a therapy for NIU may be evaluated using the following assessments.

Intraocular pressure (IOP): A Tono-Pen or Goldmann Applanation Tonometer is used to measure IOP.

Slit-lamp biomicroscopy: Slit-lamp biomicroscopy is performed using a standard slit lamp. Observations for each eye should be made for the following variables (including but not limited to): conjunctiva, cornea, lens, anterior chamber, iris, pupil, vitreous, retina, optic nerve, and fovea.

Indirect ophthalmoscopy: Dilated ophthalmoscopy is performed according to standard dilation procedures. The fundus is examined thoroughly and the following variables (including but not limited to): vitreous, retina, choroid, and optic nerve/disc.

Fluorescein angiography (FA): Anatomic assessments include the area of fluorescein leakage, area of capillary nonperfusion, the presence of retinal vascular and optic nerve head staining, and retinal pigment epithelium abnormalities.

Fundus photography: FP-4W fields (4 standard Wide Angle Fields) are used to document graded characteristics which may include vitreous haze score, lesions consistent with posterior uveitis, optic disc swelling, and vascular abnormalities.

Vitreous haze: Vitreous haze can be documented at the slit lamp using a validated scale (the SUN scale or the Miami scale) either at the slit lamp or by photographic assessment by a reading center.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The disclosure will be further clarified by the following examples, which are intended to be purely exemplary of the disclosure and in no way limiting.

### EXAMPLES

### Example 1: Clinical Evaluation of Human Anti-IL-6 Antibody in Patients with Macular Edema Due to Noninfectious Uveitis

A Phase 2 dose-ranging clinical study is designed to evaluate the efficacy and safety of human anti-IL-6 antibody TOUR006 as disclosed herein in patients with noninfectious uveitis. TOUR006 is administered as fist-line treatment of noninfectious uveitis (NIU) in patients with macular edema.

The Phase 2 study is designed to compare the efficacy and safety of two different dose arms (high dose vs. low dose) of TOUR006 vs. matching placebo (PBO) for the treatment of patients with macular edema due to noninfectious uveitis. Approximately 60 eligible patients (20 patients in each of 3 cohorts) will be randomized in a 1:1:1 ratio to SQ TOUR006 or placebo.

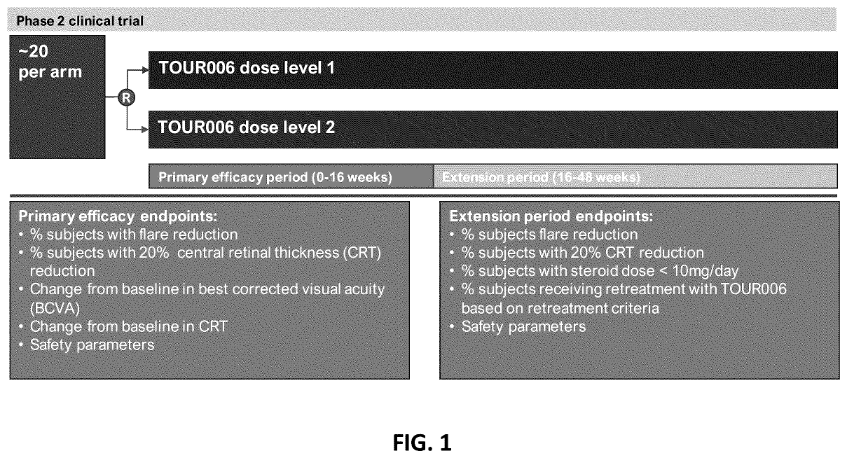

To increase flexibility and optionality, an alternative Phase 2 proof of concept (PoC) trial without placebo control is contemplated to compare a high dose arm with a low dose arm of TOUR006 (FIG. 1). A 16-week primary treatment is followed by a 32-week extension. Approximately 40 eligible patients (20 patients in each arm) will be randomized in a 1:1 ratio. The trial design parameters of the alternative design would be the same as those of the placebo-controlled design.

The unlimited examples for the high dose arm include: a) 100 mg every 8 weeks for a total of 16 weeks primary treatment; b) 50 mg every 4 weeks for a total of 16 weeks primary treatment; c) 100 mg loading dose×1, followed 4 weeks later by 50 mg every 8 weeks for a total of 16 weeks primary treatment; d) 50 mg loading dose×1, followed 4 weeks later by 50 mg every 8 weeks for a total of 16 weeks primary treatment; or e) 50 mg loading dose×1, followed 4 weeks later by 20 mg every 4 weeks for a total of 16 weeks primary treatment.

The unlimited examples for the low dose arm include: a) 50 mg every 8 weeks for a total of 16 weeks primary treatment; b) 20 mg every 4 weeks for a total of 16 weeks primary treatment; c) 50 mg loading dose×1, followed 4 weeks later by 20 mg every 4 weeks for a total of 16 weeks primary treatment; or d) 20 mg loading dose×1, followed 4 weeks later by 10 mg every 4 weeks for a total of 16 weeks primary treatment.

In some cases, the high dosing regimens may only be needed in the treatment of an acute flare of NIU. During the maintenance phase of NIU treatment (i.e., the flare has subsided but either disease is still in active phase or want to prevent the recurrence of a flare), a lower dosing regimen may be appropriate. For example, a dosing regimen for treating a patient with an acute flare of NIU may comprise a loading dose of 100 mg and a maintenance dose of 20 mg every 4 weeks. The potential duration of treatment for an acute flare may range from 1 month to as high as 12 months. The potential duration of maintenance therapy may range anywhere from 6 months to lifelong.

All patients may be allowed for steroid rescue at week 12 if worsening criteria are meet (relative to baseline).

Stratification will include CRT/CST >500 μM and BCVA <50. Treatment will continue in a masked fashion by evaluating the primary endpoint at week 16. Patients may continue in an open-label safety extension of SQ TOUR006 treatment for up to 48 weeks.

The primary objective of the study is to evaluate the efficacy of subcutaneous (SQ) TOUR006 in increasing Best Corrected Visual Acuity (BCVA) over 16 weeks in patients with macular edema due to noninfectious uveitis.

The secondary objectives of the study are: (1) to determine the anatomic effects of SQ TOUR006 in patients with macular edema secondary to noninfectious uveitis; (2) to determine the proportion of patients with visual gain in patients with macular edema secondary to noninfectious uveitis; (3) to determine the time to treatment failure in patients with macular edema secondary to noninfectious uveitis; and (4) to characterize the safety of SQ TOUR006 in patients with macular edema secondary to noninfectious uveitis.

Eligible patients with Central Retinal Thickness (CRT) or central subfield thickness (CST) >300 μM and BCVA ≥23 to ≤70 Early Treatment Diabetic Retinopathy Study (ETDRS) letters at the randomization visit will be enrolled. Patients must present with active disease and be either treatment-naive or active despite corticosteroid or disease modifying anti-rheumatic drugs (DMARD) treatment. About 40% of enroll subjects with active NIU also have macular edema (ME). Any prior treatment must not have increased for at least four weeks before randomization. DMARDS must be discontinued 48 hours before randomization. All patients will receive prednisone (or equivalent) ≥10 mg/day and <60 mg/day as background therapy at randomization. A mandatory steroid taper will start at week four.

Patients must meet all the following criteria to be included in the study:
Signed written informed consent.
≥18 years of age.
Noninfectious intermediate, posterior, or pan-uveitis in the study eye.
Active disease at screening.
Oral prednisone dose must be greater than or equal to 10 mg/day and less than 60 mg/day at randomization.
IMT stable with no increase for at least four weeks before the randomization visit.
IMT other than steroids discontinued at least 48 hours before the first study treatment injection.
CRT/CST >300 UM at randomization.
BCVA ≥23 to ≤70 ETDRS letters at randomization.
Best-corrected visual acuity of 20/400 or better in the fellow eye (approximately 20 letters).
Chest radiograph within three months of enrollment with no evidence of malignancy, infection, or fibrosis.
Females of childbearing potential must have a negative serum pregnancy test at screening. In addition, sexually active females of childbearing potential must agree to use TWO of the following adequate forms of contraception while on study medication: oral, injectable, or implantable hormonal contraceptives; tubal ligation; intrauterine device; barrier contraceptive with spermicide; or vasectomized partner.
Males must agree to use barrier contraception (latex condoms) when engaging in sexual activity while on study medication and for 28 days after taking the last dose of study medication.

Patients must not enter the study if any of the following exclusion criteria is present:

Participants with confirmed or suspected uveitis of infectious etiology or uveitis of traumatic etiology.
Prior treatment with anti-interleukin-6 (IL-6) or interleukin-6 receptor complex (IL-6R) antagonist therapies, including tocilizumab and sarilumab.
Suspected/confirmed central nervous system or ocular lymphoma.
Uncontrolled glaucoma.
Use of topical ocular medication other than lubricating eye drops.
Implanted device.
Significant ocular disease
Lens/media opacities or obscured ocular media.
Intraocular surgery or treatments.
Capsulotomy within 6 months of randomization.
Ocular or periocular infection.
History of herpetic infection.
Toxoplasmosis or toxoplasmosis scar.
Ocular malignancy.
Allergy or hypersensitivity to study drug.
Participation in other uveitis trials within 30 days.
Any systemic condition/infection.
Immunocompromised.
History of GI perforation or bleed.
Planned elective ocular surgery during the first six months of the study.

The study will consist of three phases: (i) screening and randomization, (ii) treatment, and (iii) follow-up. During the screening, each potential subject will provide informed consent before starting any study-specific procedures. The randomization of subjects to study groups will be performed centrally by an Interactive Web-Response System (IWRS) using a randomization scheme reviewed and approved by an independent statistician. During the treatment period, randomized subjects will be provided the treatment and assessment according to the protocol. Follow-up will occur 28-days following termination of the IP. In Patients will continue their randomized treatment unless treatment failure occurs. At week 8, patients who fail treatment will be eligible for rescue with SQ TOUR006 50 mg every 4 weeks in an open-label arm until the end of the study. Treatment failure is defined as a loss of ≥10 letters of BCVA, CRT/CST increase of 20%, a 2-step increase in vitreous haze or anterior chamber cells (Standardization of Uveitis Nomenclature (SUN) classification), or the occurrence of increased retinal vascular lesion compared with baseline.

If any of the following criteria is met in the study eye at week 4 or week 8, retreatment with TOUR006 is required.
Macular edema or subretinal fluid (new or persistent) in conjunction with a CRT or CST >340 μM as measured by OCT.
A decrease in BCVA of 10 letters (ETDRS) or greater between the current visit and the BCVA reading from the previous visit.
A decrease in BCVA of 10 letters (ETDRS) or greater from the best measurement (during the study) with an increase in CRT or CST of >50 μM from the previous visit, associated with new fluid.

The primary efficacy endpoint includes: 1) % subjects with flare reduction including 2-step reduction in vitreous haze, reduction in retinal lesions, reduction in retinal vessel leakage, and reduction in anterior chamber cells; 2) % subjects showing 20% CRT or CST reduction from baseline; 3) a change from baseline in BCVA; 4) a change from baseline in CRT or CST; and 5) changes in safety parameters including IOP, slit lamp biomicroscopy, indirect ophthalmoscopy, fluorescein angiography (FA), OCT, and fundus photography. In one embodiment, the probability of vitreous haze reduction from baseline is at least about 50%, about 60%, about 70%, about 80%, or about 90%. In one embodiment, the probability of CRT or CST reduction from baseline is at least about 50%, about 60%, about 70%, about 80%, or about 90%. In another embodiment, CRT or CST is reduced to <300 μM.

The extend period endpoints include: 1) % subjects with flare reduction; 2) at least 50% subjects showing 20% CRT or CST reduction from baseline; 3) % subjects with steroid dose <10 mg/day; 4) % subjects receiving retreatment with TOUR006 based on retreatment criteria; and 5) incidence of changes in safety parameters, including IOP, slit lamp biomicroscopy, indirect ophthalmoscopy, fluorescein Angiography (FA), OCT, and fundus photography. In one embodiment, the probability of flare reduction from baseline is at least about 50%, about 60%, about 70%, about 80%, or about 90%. In one embodiment, the probability of CRT or CST reduction from baseline is at least about 50%, about 60%, about 70%, about 80%, or about 90%. In one embodiment, the probability of reducing steroid to less than 10 mg/day is at least about 50%, about 60%, about 70%, about 80%, or about 90%. In one embodiment, the probability of receiving retreatment with TOUR006 is no more than about 50%, about 40%, about 30%, about 20%, or about 10%.

Other secondary endpoints may include: (a) the proportion of evaluable patients who show improvement in retinal vascular lesions at week 16; (b) the proportion of evaluable eyes with a 2-step improvement in vitreous haze at week 16; (c) the proportion of eyes that gain 10 ETDRS letters from baseline at week 16; (d) proportion of eyes that gain 15 letters from baseline at week 16; (e) the proportion of patients that met criteria for treatment failure at week 16; (f) the change from baseline in the National Eye Institute Visual Function Questionnaire-25 (NEI VFQ-25) near activities subscale score at week 16; (g) change from baseline in the NEI VFQ-25 distance activities subscale score at week 16; and (h) a safety analysis of TOUR006.

Safety may be assessed by pre- and post-treatment serial measurements of vital signs, clinical laboratory assessments, and the recording of adverse clinical events. Changes in the clinical status of the uveitis may be evaluated to gauge long-term and average disease control, using an approach described in Sfikakis et al. (Lancet 28:358, 2001), the contents of which are hereby expressly incorporated by reference in their entirety for any purpose.

Both primary and secondary efficacy endpoints will be evaluated at a 2-sided significance level of 2.5% (Bonferroni adjustment for the 2 comparisons on the primary efficacy endpoint). Continuous variables will be analyzed with an analysis of covariance with the baseline value as covariate and treatment group as fixed factors. Proportions will be analyzed using a Cochran Mantel-Haenszel test stratified by BCVA and CRT/CST. Both primary and secondary efficacy endpoints will be evaluated at a 2-sided significance level of 2.5%. Missing values will be imputed using the last observation carried forward (LOCF) method. Safety will include all randomized patients who receive any study treatment.

### Example 2. PK/PD Modeling of TOUR006

The purpose of this study is to inform dosing parameters of TOUR006 for the treatment of NIU using pharmacokinetic/pharmacodynamic (PK/PD) based simulations.

C-reactive protein (CRP) is directly downstream of IL-6 signal transduction, and irrelevant if ligand or receptor is blocked. There is tightly linked temporal association between CRP and IL-6 pathway. Thus, serum CRP is a promising pharmacology marker of IL-6 pathway activity. Using CRP as marker to identify what level of IL-6 pathway suppression associated with the dosing regimen would inform PD goal of TOUR006 for the treatment of NIU.

Population modeling analyses were conducted using nonlinear mixed effects modeling. The CRP data for the modeling came from a multiple dose study of TOUR006 in rheumatoid arthritis (RA) patients receiving background methotrexate (NCT00838565) and the clinical investigation of tocilizumab in RA patients (Paccaly et al., J Clin Pharmacol. 2021 January; 61 (1): 90-104; Xu et al., J. of Clinical Pharma., 2021, 61 (5): 714-724.) The assumption of the modeling is that the background inflammatory state of NIU is similar to RA. The published observations of CRP levels in NIU are consistent with the model's assumptions. For example, the mean and median concentrations of hsCRP in patients with NIU are less than 10 mg/L, with many patients having less than 5 mg/L. (Ozgonul et al., Int J Ophthalmol. 2017; 10 (2): 262-266.).

The PK/PD model explored two subpopulations with relatively less severe and more severe inflammation:
Population A: Baseline CRP >2 mg/L to 10 mg/L
Population B: Baseline CRP >10 mg/L.

The PK/PD based simulations were performed for the dosing scenarios given in Table 2.

TABLE 2
Dosing regimen

50 mg LD, followed by 20 mg Q4W starting at 4 weeks
20 mg LD, followed by 10 mg Q4W starting at 4 weeks
50 mg Q8W
20 mg Q8W
50 mg Q12W
30 mg Q12W
20 mg Q12W
50 mg LD, followed by 50 mg Q12W starting at 4 weeks
20 mg LD, followed by 30 mg Q12W starting at 4 weeks
Note:
“LD” refers to loading dose;
“Q” herein refers to “every”; and
“W” refers to “week”

The simulation results were calculated for 4, 8, 12, 16, 20, and 24 weeks from the first dose.

The CRP suppression goal is at least 90% decrease from baseline (based upon CRP effects observed from Tocilizumab 8 mg/kg IV q4 weeks in RA). The simulations modeled what percentage of patients attained the CRP suppression goal for a given TOUR006 regimen. Any patient with CRP suppression of less than 2 mg/L after 7 days of treatment was considered to have at least 90% suppression. This was done to avoid ceiling effects (<2 mg/L is entering into the normal range).

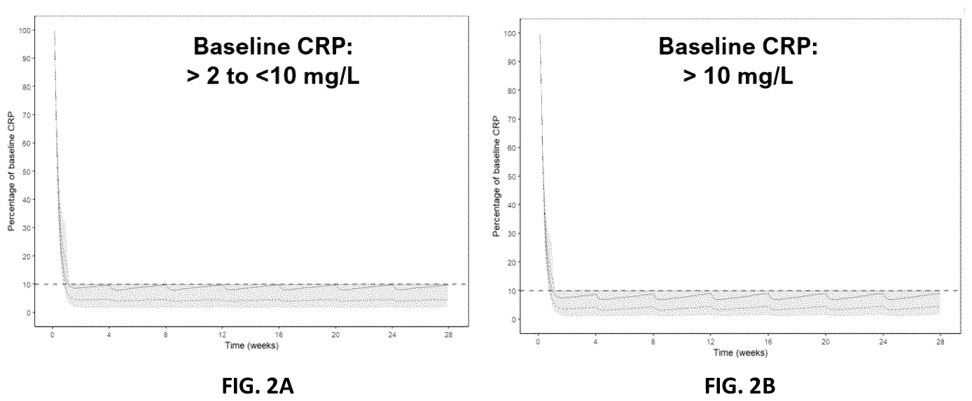

As shown in FIG. 2A and FIG. 2B, the PK/PD modeling predicts that nearly all patients, including population A (FIG. 2A) and population B (FIG. 2B), rapidly attain the CRP suppression goal (≥90% decrease) under the dose regimen of 50 mg LD, followed by 20 mg Q4W starting at 4 weeks.

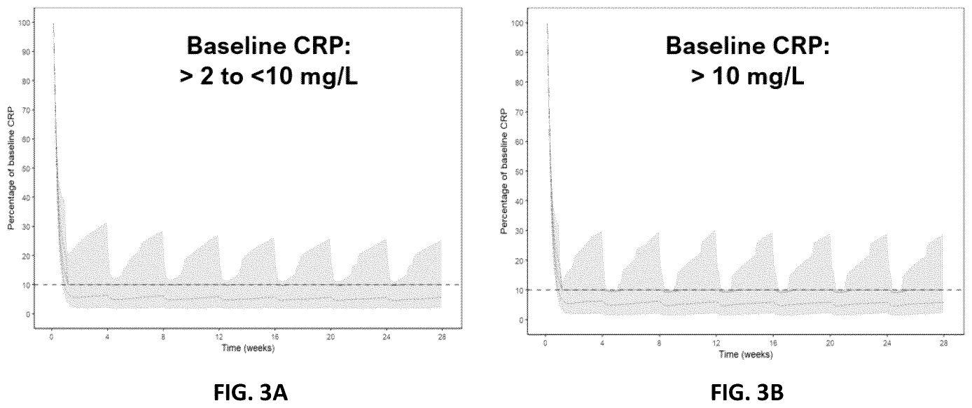

As shown in FIG. 3A and FIG. 3B, the PK/PD modeling predicts that most patients (~90%), including population A (FIG. 3A) and population B (FIG. 3B), rapidly attain the CRP suppression goal (≥90% decrease) under the dose regimen of 20 mg LD, followed by 10 mg Q4W starting at 4 weeks, although with incomplete population coverage relative to the dose regimen of 50 mg LD, followed by 20 mg Q4W starting at 4 weeks.

Further, the PK/PD modeling predicts rapid and robust CRP suppression for both dose regimens-50 mg LD, followed by 20 mg Q4W starting at 4 weeks, and 20 mg LD, followed by 10 mg Q4W starting at 4 weeks. Table 3 and Table 4 provide the percentage of patients with at least 90% CRP suppression over the treatment period.

TABLE 3
Percent of patients with ≥90% CRP suppression under the dose
regimen of 50 mg LD, followed by 20 mg Q4W starting at 4 weeks

| Baseline | Week | Week | Week | Week | Week | Week |
|---|---|---|---|---|---|---|
| CRP | 4 | 8 | 12 | 16 | 20 | 24 |
| >2 to ≤10 | 94% | 94% | 94% | 94% | 94% | 94% |
| mg/L | | | | | | |
| >10 mg/L | 97% | 96% | 96% | 95% | 95% | 95% |

TABLE 4
Percent of patients with ≥90% CRP suppression under the dose
regimen of 20 mg LD, followed by 10 mg Q4W starting at 4 weeks

| Baseline | Week | Week | Week | Week | Week | Week |
|---|---|---|---|---|---|---|
| CRP | 4 | 8 | 12 | 16 | 20 | 24 |
| >2 to ≤10 | 89% | 90% | 90% | 91% | 91% | 91% |
| mg/L | | | | | | |
| >10 mg/L | 89% | 89% | 89% | 89% | 89% | 90% |

Additionally, the PK/PD modeling predicts that less frequent dosing regimens achieve the CRP suppression goal of at least 90% decrease from baseline within the 24 weeks treatment period. Tables 5 to 7 provide the percentage of patients with at least 90% CRP suppression over the treatment period under the less frequent dosing regimens.

TABLE 5
Percent of patients with ≥90% CRP suppression
under the dose regimen of 50 mg Q8W

| Baseline | Week | Week | Week | Week | Week | Week |
|---|---|---|---|---|---|---|
| CRP | 4 | 8 | 12 | 16 | 20 | 24 |
| >2 to ≤10 | 98% | 95% | 98% | 97% | 98% | 97% |
| mg/L | | | | | | |
| >10 mg/L | 95% | 93% | 97% | 95% | 98% | 95% |

TABLE 6
Percent of patients with ≥90% CRP suppression
under the dose regimen of 20 mg Q8W

| Baseline | Week | Week | Week | Week | Week | Week |
|---|---|---|---|---|---|---|
| CRP | 4 | 8 | 12 | 16 | 20 | 24 |
| >2 to ≤10 | 94% | 88% | 94% | 93% | 95% | 93% |
| mg/L | | | | | | |
| >10 mg/L | 91% | 80% | 93% | 85% | 94% | 87% |

TABLE 7
Percent of patients with ≥90% CRP suppression
under the dose regimen of 50 mg Q12W

| Baseline | Week | Week | Week | Week | Week | Week |
|---|---|---|---|---|---|---|
| CRP | 4 | 8 | 12 | 16 | 20 | 24 |
| >2 to ≤10 | 99% | 99% | 94% | 100% | 98% | 94% |
| mg/L | | | | | | |
| >10 mg/L | 99% | 96% | 94% | 99% | 96% | 94% |

The PK/PD modeling predicts effective dosage arms for NIU Phase 2 trial. Specifically, a dosing regimen of 50 mg LD, followed by 20 mg Q4W starting at 4 weeks is predicted to result in 94-98% of patients to achieve target CRP suppression in both the moderate inflammation (i.e., baseline CRP 2 to 10 mg/L) and severe inflammation (i.e., baseline CRP of >10 mg/L) populations. A dosing regimen of 20 mg LD, followed by 10 mg Q4W starting at 4 weeks is predicted to result in ~90% of patients to achieve target CRP suppression in both the moderate inflammation and severe inflammation populations. Both regimens are predicted to achieve rapid (i.e., in 2 weeks or less) suppression of CRP by >90% from baseline.

The PK/PD modeling predicts that less frequent dosing regimens offer opportunity for robust CRP suppression while further reducing drug administration burden for patients. Specifically, the less frequent dosing regimens including 50 mg Q8W and 50 mg Q12W regimens are both predicted to offer CRP suppression effects similar to the 50 mg LD, followed by 20 mg Q4W starting at 4 weeks regimen. The 20 mg Q8W regimen is predicted to offer CRP suppression effects similar to the regimen of 20 mg LD, followed by 10 mg Q4W starting at 4 weeks.

The PK/PD modeling results predict that less frequent or lower dose regimens offer similar CRP suppression effects, particularly if a loading dose is used.

NIU tends to have more severe inflammation than other ocular diseases such as thyroid eye disease (TED) or Myasthenia Gravis (MG), either at the baseline state for most patients or in the context of periodic flares that may intermittently surge to higher levels of inflammation. Without being bound by a theory, it is thought that high levels of inflammation observed in NIU may be, at least in part, due to a higher level of IL-6 pathway activation. Therefore, higher drug exposure may be needed to further suppress IL-6 pathway activity in patients with NIU beyond the individual patient goal of ≥90% CRP decrease from baseline. Additionally, the risk for blindness may warrant erring on the side of higher pharmacologic coverage (than what may be needed to attain ≥90% CRP decrease from baseline in most patients). Higher drug exposure can be achievable through higher dose levels, more frequent dosing intervals and/or use of a loading dose or loading regimen as described in the foregoing Example 1 of Phase 2 clinical trial for NIU.

Overall, the PK/PD Modeling predicts that the planned dosing regimens of TOUR006 offer broad, deep and durable effects, an appropriate safety profile and low drug administration burden which supports a patient-centric treatment experience.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method of treating noninfectious uveitis (NIU) comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10.

2. The method of claim 1, wherein the anti-IL-6 antibody or antibody fragment comprises a heavy chain polypeptide comprising a polypeptide having at least 98% identity to SEQ ID NO: 1 and a light chain polypeptide comprising a polypeptide having at least 98% identity to SEQ ID NO: 7.

3. The method of claim 1, wherein the anti-IL-6 antibody or antibody fragment comprises a heavy chain polypeptide having the sequence of SEQ ID NO: 1 and a light chain polypeptide having the sequence of SEQ ID NO: 7.

4. The method of claim 1, wherein the anti-IL-6 antibody or antibody fragment containing said CDRs is contained in a pharmaceutical composition that comprises said anti-IL-6 antibody or antibody fragment and a pharmaceutically acceptable carrier.

5. The method of claim 1, wherein said therapeutically effective dose is administered subcutaneously.

6. The method of claim 1, wherein said therapeutically effective dose is between 5 mg to 200 mg.

7. The method of claim 1 wherein said therapeutically effective dose is administered from every week to every 24 weeks.

8. The method of claim 6, wherein said therapeutically effective dose is 100 mg and is administered every 8 weeks.

9. The method of claim 6, wherein said therapeutically effective dose is 50 mg and is administered every 4 weeks.

10. The method of claim 6, wherein said therapeutically effective dose is 50 mg and is administered every 8 weeks

11-22. (canceled)

23. The method of claim 1, further comprising administering a steroid to the patient.

24-28. (canceled)

29. The method of claim 1, wherein the patient is at least 18 years old.

30-32. (canceled)

33. The method of claim 1, the patient has macular edema (ME) in anterior uveitis that is unresponsive to topical steroids.

34. The method of claim 1, wherein the patient has stable immunomodulatory therapy (IMT) with no increase for at least four weeks before the first dose.

35-36. (canceled)

37. The method of claim 1, wherein the patient has best corrected visual acuity (BCVA) from at least 23 to no more than 70 Early Treatment Diabetic Retinopathy Study (ETDRS) letters or Snellen equivalent.

38-41. (canceled)

42. The method of claim 1, wherein the patient does not experience:
(a) confirmed or suspected uveitis of infectious etiology or uveitis of traumatic etiology;
(b) confirmed or suspected central nervous system or ocular lymphoma;
(c) uncontrolled glaucoma;
(d) a significant ocular disease;
(e) an ocular or periocular infection;
(f) a history of herpetic infection;
(g) toxoplasmosis or toxoplasmosis scar;
(h) ocular malignancy;
(i) a systemic infection;
(j) immunocompromised;
(k) a history of gastrointestinal perforation or bleed; or
(l) allergy or hypersensitivity to the anti-IL-6 antibody or antibody fragment.

43. The method of claim 1, wherein the patient does not receive:
(a) prior treatment with an anti-IL-6 or IL-6 receptor complex (IL-6R) antagonist therapy;
(b) a topical ocular medication other than lubricating eye drops;
(c) an implanted device;
(d) lens/media opacities or obscured ocular media;
(e) intraocular surgery or treatment;
(f) capsulotomy within 6 months of the first dose.

44. The method of claim 42, wherein the anti-IL-6 or IL-6R antagonist therapy comprises tocilizumab and sarilumab.

45. The method of claim 1, wherein the patient is treatment-naïve or active despite corticosteroid or disease modifying anti-rheumatic drugs (DMARD) treatment.

46. The method of claim 1, wherein said method of treatment achieves one or more of the following results:
(a) a flare reduction;
(b) at least about 20% reduction in Central Retinal Thickness (CRT) or central subfield thickness (CST) in eyes with macular edema (CRT or CST >300 μm) when measured after the start of treatment;
(c) an increase in best corrected visual acuity (BCVA) of the patient by at least 10 letters or Snellen equivalent when measured after the start of treatment, or
(d) quiescence with a total steroid dose of <10 mg/day.

47-52. (canceled)


SEQUENCE LISTING (12 sequences)

SEQ ID NO: 1 - protein, 442 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 5 aa, Homo sapiens
EYYWS

SEQ ID NO: 3 - protein, 16 aa, Homo sapiens
EIFHSGSTNYNPSLKS

SEQ ID NO: 4 - protein, 8 aa, Homo sapiens
EELDDFDI

SEQ ID NO: 5 - protein, 116 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSS

SEQ ID NO: 6 - DNA, 1326 bp, Homo sapiens
  source 1..1326: mol_type = other DNA
caggtgcagctacagcagtggggcgcaggactgttgaagccttcggagaccctgtccctcacctgcgctatctatggtgggtccttcagggagtactactggagctggatccgccagcccccagggaaggggctggagtggattggggaaatctttcatagtggaagcaccaactacaacccgtccctcaagagtcgagtcaccatatcagtagacacgtccaagaaccagttctccctgaagctgagctctgtgaccgccgcggacacggctgtgtattactgtgcgagagaggaattagatgattttgatatctggggccaagggacaatggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtagtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYKSYPRT

SEQ ID NO: 11 - protein, 107 aa, Homo sapiens [sequence omitted]

SEQ ID NO: 12 - DNA, 642 bp, Homo sapiens
  source 1..642: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagtagcctgcagcctgaagattttgcaacttattactgccaacagtataaaagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt